(19) United States
(12) Patent Application Publication
(10) Pub. No.: US 2011/0015487 A1
(43) Pub. Date: Jan. 20, 2011

(54) ENDOSCOPE WITH SINGLE-CHANNEL ENDOSCOPIC TUBE AND DISPOSABLE SET, CONNECTING THE CHANNEL WITH PRESSURE SOURCES.

(76) Inventor: Sergey Matasov, Riga (LV)

Correspondence Address: Sergey Matasov, Ranka Bambis 7/1-55, Riga, LV 1048, LV

(21) Appl. No.: 12/922,851

(22) PCT Filed: Mar. 21, 2009
(86) PCT No.: PCT/IB2009/005056

§ 371 Date: Sep. 16, 2010

(30) Foreign Application Priority Data
Mar. 20, 2008 (LV) 9-08-44

(51) Int. Cl. A61B 1/00

(52) U.S. Cl. 600/153

(57) ABSTRACT

An aim of the invention is to provide a safe, quick and cheap reuse of an endoscopic tube, simplification of its construction; the technical task is creation of an endoscope with an endoscopic tube without cavities which require complicated washing.

The endoscope comprises:
an endoscopic tube, comprising an instrumental channel with a lateral output directed on an objective lens and with a diameter of a direct output close to the diameter of the distal part of instrument,
an external tubular element in a shape of a tube, where one end is bifurcated and adjusted for connection with an instrumental channel and for introduction of instrument, and the other end is branched and adjusted for connection with pressure sources,
valves which squeeze the lumens of branches, but at switching on—release them.

On the drawing are represented:
a handle (1) of a valve-less single-channel endoscopic tube,
a tubular element (15, 16, 17) with a block of valves (21),
places of connection of the element with a channel (5) and with a pressure sources.

The tubular element, valves, clips (25) compose a disposable sterile set. The valve-less single-channel endoscopic tube and the set provide all endoscopic functions. Its washing will become safe, quick and cheap, and its manufacturing and reprocessing will be reduced in price as well.

### TECHNICAL FIELD

The usage area of the invention—medical and veterinary endoscopy, the matter of invention—an endoscope. The concrete aim of the matter of invention—to ensure safe, quick and cheap reuse of an endoscopic tube, moreover—to simplify its construction. In some cases the invention would be useful for the industrial endoscopy.

### BACKGROUND

Among the basic functions of medical endoscopes in addition to the visual one, which comprises illumination and transferring of image of walls of the investigated cavity, there are also:
feeding of gas or liquid into cavity,
aspiration of cavity,
washing of objective lens,
biopsy of walls of the investigated cavity and others instrumental manipulations.

An endoscope comprises an endoscopic tube, which consists of communication part and insertion part, interconnected by a handle. The length of an endoscopic tube could be 3.2 meters long and more. The above mentioned functions are ensured by:
channels (see Endoscope Reprocessing: Current Status of Disinfection Recommendations; William A. Rutala, Ph. D. M.P.H. (www.unc.edu/depts/spice/dis/endoscope.ppt, slide 10), comprised in the communication part and connected with the pressure sources on its free end,
valves, comprised in the handle, which interconnect the channels of the communication part and the insertion part,
channels, comprised in the insertion part, which are opened into the investigated cavity, and among them—an instrumental channel with an entry on the handle.

### TECHNICAL PROBLEM

Endoscopes are expensive and reused; the more frequent—the higher is its economical efficiency. The usage frequency depends on the speed of reprocessing of an endoscopic tube: washing, disinfection and, in case of necessity, sterilization. An endoscopic tube is a potential spreader of infectious hepatitis, AIDS and others infection diseases. The locating places of the blood, tissues, biology secret, contents of the investigated organ of previous patient, are channels and valves of an endoscopic tube.

The key moment of reprocessing is washing. The outward surface of an endoscopic tube is washed easily. The valves are washed in disassembled state because of their complicated configuration, presence of angular, blind and buried cavities. An instrumental channel (its diameter is about 3 mm) is brushed and washed by liquid flow. Into channels with diameter 1.2 mm, intended for feeding of gas and liquid, is impossible to introduce a brush. Speed of their washing depends on a speed of the washing liquid flow, but the flow speed depends on its pressure, however the pressure over 0.2 bar is impermissible, as it can disturb hermecity of the system.

The punctual compliance with the manual washing technologies requires a lot of time, but its simplification threatens by a cross-infection of patients, damage of endoscopic tube. Washing machines are expensive and their usage does not shorten the time of reprocessing for much. As a result the following conclusion could be made: an endoscopic tube's washing is a technical problem.

The problem of washing of channels with diameter 1.2 mm is illustrated by a number of articles, here is a conclusion from one of them: “Therefore, a redesign of the fundamental structure of endoscopes is proposed to all manufacturers in order to make air and water channels accessible for brushing and to reduce a risk of intra-hospital infections” (ISHIDO Yumiko; IDO Kenichi; KOIWAI Hirobumi; SUGANO Kentaro; Gastrointestinal endoscopy, 2001, vol. 53, n^{o}2, pp. 165-168 (23 ref.).

The present invention excludes the recommended way.

### DISCLOSURE OF INVENTION

The technical problem has outlined the purpose and the task of modification of the closest prototype of the present invention. It should be an endoscope which comprises:
pressure sources,
an endoscopic tube, which comprises channels and valves that interconnect the pressure sources with an investigated cavity, and among them—the instrumental channel.

The purpose of the present invention—to provide a safe, quick and cheap reuse of an endoscopic tube, moreover—a simplification of its design, the technical task—to create an endoscope with an endoscopic tube without cavities requiring a protracted and complicated reprocessing. The technical result of solving of this task will be an endoscope, comprising:
pressure sources,
an endoscopic tube, comprising an instrumental channel with a lateral output directed on the objective lens and with a diameter of a direct output close to the diameter of the distal part of instrument,
an external tubular element in a shape of a tube, where one end is bifurcated and adjusted for connection with an instrumental channel and for introduction of instrument, and the other end is branched and adjusted for connection with pressure sources,
valves, which at switching off squeeze the lumens of branches of tubular element, but at switching on—release them,
a bung of the direct output from an instrumental channel,
a bung of the place for introduction of instrument into the tubular element.

Here is assignment of the above listed elements:
An instrumental channel of the endoscopic tube is universal; it interconnects the investigated cavity with any of pressure sources. At the diameter of a channel and an instrument, for example, of 4 mm and 2.3 mm accordingly, the area of gap between them will correspond to the area of a tube with diameter 3 mm. It is enough for decompression of the investigated cavity, for feeding inside it of gas or liquid without extraction of instrument. The lateral output of channel is intended for cleaning of objective lens, at that the direct output should be closed by instrument or by bung.
A tubular element is a disposable part of the system, that is—it is intended only for one patient. Taking into account its simple design, a cost of tubular element will be rather small. It could be made, for example, from silicon.
Valves allow to connect selectively one or another source of pressure with an universal instrumental channel. The advantage of squeezing valves is, that they do not disturb an integrity and hermecity of tubular element. In the optimal variant the valves will be key-shaped and placed as a block on the handle of an endoscopic tube. The pedal-shaped valves are also permissible.
Bungs of an universal instrumental channel could be used for endoscopy of gastrointestinal tract when there is a prognosis, that biopsy and other instrumental operations will not be needed. If this supposition will not justify oneself, the bung of the entry into a channel is removed, and the bung of output is squeezed out by an instrument inside the tract.

For fixing of branches of the tubular element to the communication part of the endoscopic tube the clips could be used. The simple design of the tubular element, of the block of valves and of the clips allows to unite them into a disposable set.

Washing of an endoscopic tube which has no valves and has a single channel with a large of cross-section area (for example, for diameter 4 mm it is 11 times larger than the cross-section area of channel with diameter 1.2 mm), will occupy only few minutes.

This invention will exclude the necessity to buy expensive washing machines and thus will substantially reduce the price of equipment of endoscopic cabinets.

The invention will influence also on the cost of endoscopes: manufacturing and repair of valve-less single-channel endoscopic tube is perceptibly cheaper, than of existing ones.

### BRIEF DESCRIPTION OF DRAWINGS

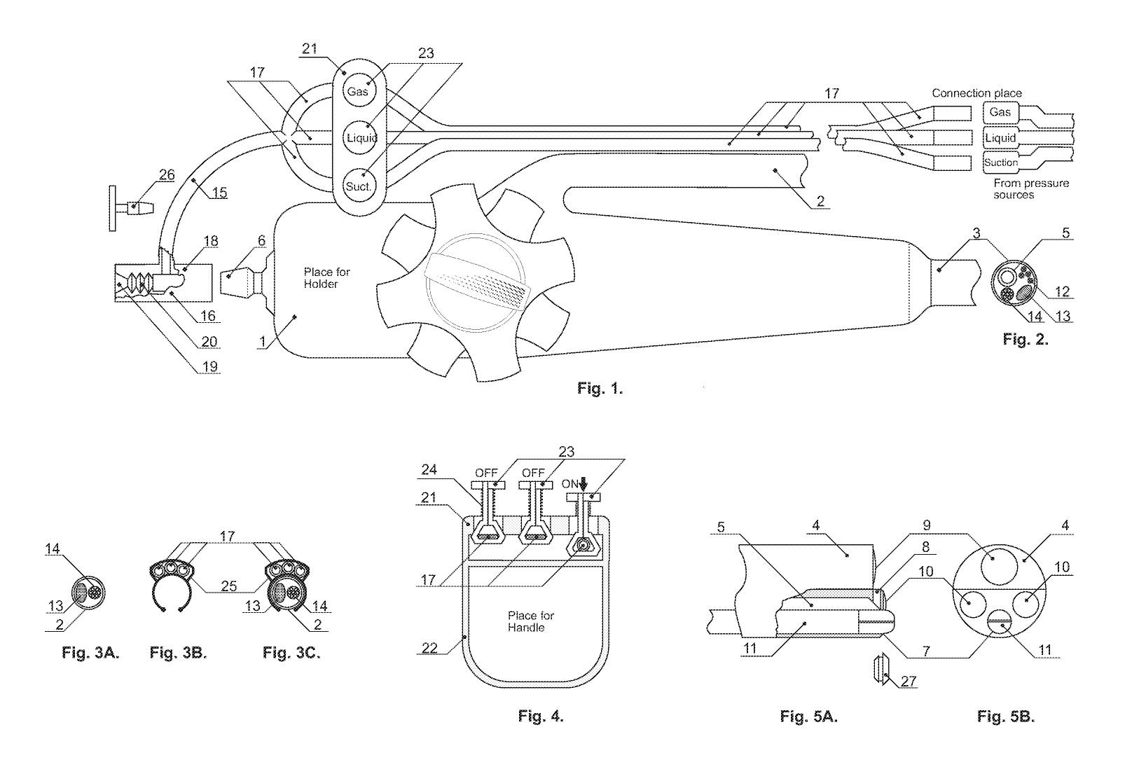

The invention is illustrated by the drawing 1/1, where in a scale 1:1 (on FIGS. 1-4) and 2.5:1 (on FIG. 5A, 5C) are represented:

on FIG. 1—a handle of an endoscopic tube, a tubular element with a block of valves, the places of connection of a tubular element with an universal instrumental channel and with tubes of pressure sources;

on FIG. 2—a cross-section of the insertion part of an endoscopic tube;

on FIG. 3A—a cross-section of the communication part of an endoscopic tube;

on FIG. 3B—a cross-section of branches of a tubular element and of clips, which support it;

on FIG. 3C—a cross-section of branches of a tubular element, of clips and a communication part of an endoscopic tube as assembled;

on FIG. 4—a longitudinal section of a block of valves;

on FIG. 5A—an end-view of the distal head of the insertion part with an introduced instrument;

on FIG. 5B—a butt end of the distal head of the insertion part with an introduced instrument.

### BEST MODE FOR CARRYING OUT THE INVENTION

The basic parts of construction are an endoscopic tube, a tubular element, a block of valves. The endoscopic tube (note FIG. 1, 2, 3A, 5A, 5C) comprises:
1—a handle,
2—a communication part,
3—an insertion part,
4—a distal head of the part 3,
5—an universal instrumental channel,
6—a fitting of the channel 5,
7—a direct output of the channel 5,
8—a lateral output of the channel 5,
9—an objective lens,
10—lenses of light-pipe,
1—an instrument,
12—units Bowden-wire,
13—a light-pipe,
14—a CCD cable.

The tubular element comprises:
15—a central tube,
16—a bifurcation,
17—branches,
18—a place of connection with the fitting 6 of the channel 5,
19—a place of introduction of instrument,
20—a seal of instrument.

The block of valves 21 comprises:
22—a holder on the handle 1,
23—valves with springs 24.

The tubular element with the block of valves 21 putted on its branches 17 (note FIGS. 1 and 4) and with clips 25 (note FIG. 3B and 3C), which support branches 17 on the communication part 2 of the endoscopic tube, will compose a disposable sterile set. The bungs 26, 27 of the entrance 19 and the direct output 7 of the channel 5 could be additionally included in the sets for gastroenterological endoscopes.

The process of preparation for endoscopy comprises:
putting of the block of valves 21 on the handle 1 (note FIG. 1 and FIG. 4),
putting of the clips 25 on the communication part 2 (note FIG. 3A, 3B, 3C),
putting of the end 18 of the tubular element on the fitting 6 (note FIG. 1),
connection of ends of branches 17 with the pressure sources (note FIG. 1),
closing of entrance 19 and the output 7 by the bungs 26, 27.

In case of use of bungs 26, 27 the blowing, decompression of the investigated cavity, cleaning of the objective lens 9 are carried out as previously—by pressing the corresponding button on the block 21. For instrumental interference, for example biopsy, is necessary to extract the bung 26, to introduce the instrument 11 through the entrance 19 into the channel 5 and to squeeze out the bung 27 by it. In future all endoscopic functions are also realized as usually, taking into account that the lens 9 is necessary to be cleaned at the closed output 7 (note FIG. 5A and 5B), but the aspiration—at the opened one.

It is necessary to emphasize, that the bung 27 is intended for endoscopes for gastrointestinal tract, as only in this case it can safely go out in a natural way.

After examination the endoscopic tube is to be released from the disposable set and is necessary to immediately proceed with its hand washing. The channel 5 is washed through the fitting 6 by the liquid flow and by brush. The quick and safe reprocessing and disinfection of the tube will allow already after 15 minutes to reuse it for a next patient.

1. An endoscope, comprising pressure sources and an endoscopic tube, which comprises an instrumental channel (5), wherein the instrumental channel (5) has a lateral output (8) on an objective lens (9), and the diameter of a direct output (7) is close to the diameter of the distal part of an instrument (11).

2. The endoscope according to the claim 1, which additionally comprises an external tubular element in a shape of a tube (15) where one end is bifurcated (16, 18, 19) and adjusted for connection with an instrumental channel (6, 5) and for introduction of the instrument (11), and the other end is branched (17) and adjusted for connection with pressure sources.

3. The endoscope according to the claim 2, which additionally comprises valves (23), which at switching off squeeze the branches (17) of the tubular element, but at switching on—release them.

4. The endoscope according to the claim 2, wherein the instrumental channel (5) has a bung (27) of the direct output (7).

5. The endoscope according to the claim 2, wherein the tubular element has a bung (26) of a place (19) of introduction of the instrument (11).
